(19) United States
(12) Patent Application Publication
(10) Pub. No.: US 2003/0192736 A1
(43) Pub. Date: Oct. 16, 2003

(54) Dual passage binaural stethoscope

(76) Inventor: Marc S. Werblud, Seattle, WA (US)

Correspondence Address: Robert W. Jenny, Apt. 313, 319 - 101st SE, Bellevue, WA, 98004, US

(21) Appl. No.: 10/122,903

(22) Filed: Apr. 16, 2002

(51) Int. Cl. H04R 25/02; A61B 7/02

(52) U.S. Cl. 181/131; 181/130

(57) ABSTRACT

The stethoscope has a chest piece and two earpieces and two continuous passages, one from each earpiece to the chest piece. The passages may be in separate tubes which may be connected side-by-side over a portion of their lengths or the passages may be in a single part called a passage member which is bifurcated to allow one passage to go to each earpiece. The earpieces and portions of the tubes or passage member are attached by a leaf spring which positions the earpieces relative to each other.

### BACKGROUND OF THE INVENTION

1. Field

The subject invention is in the field of equipment and apparatus for transmission of acoustic energy (sound) from a specific source to both of a user's ears. More specifically, it is in the field of such equipment and apparatus in which the transmission is pneumatic (i.e. air passages). Well known apparatus in this specific field includes the headsets used in the sound systems on commercial aircraft and stethoscopes.

2. Prior Art

There is much prior art in this specific field, patented and not, primarily related to acoustic headsets using pneumatics and stethoscopes. Some of these headsets use two tubes, one for each ear. The tubes are connected to separate sound sources to provide stereophonic sound to users. Each tube is connected to an earpiece. The earpieces may or may not be interconnected by a spring assembly or similar structure. This configuration, for purposes of this disclosure, is termed “dual-tube stereophonic”.

In conventional stethoscopes the sounds to be transmitted to the earpieces are picked up by the diaphragm of a chest piece of a stethoscope. The chest piece is connected to a tube which is bifurcated at a point along its length to provide two tubes, each connected to an earpiece. This configuration, for purposes of this disclosure, is termed “bifurcated tube binaural”. At the bifurcation the cross sectional area of the passage for the sounds changes in a short, even very short, distance. For example, if the cross sectional areas of the two tubes are each equal to that of the single tube, the cross sectional area is doubled. For a second example, if the combined cross sectional areas of the two tubes is made equal to the cross sectional area of the single tube, there is a sudden change in the diameter of the sound passages. It is considered that these abrupt changes in the sound passageway have deleterious effects on the quality and/or fidelity of the sound transmission such that it is not equivalent to that of dual tube stereophonic equipment.

Accordingly, the primary objective of the subject invention is to provide a stethoscope having dual passage, binaural, pneumatic sound transmission. Other objectives are that the stethoscope be competitive in terms of performance, weight, durability, cost to manufacture and professional quality and appearance.

### SUMMARY OF THE INVENTION

The subject invention is a dual passage binaural stethoscope. It comprises primarily a conventional chest piece, two earpieces or earpiece assemblies, hereafter in this disclosure termed earpieces, an elongated passage member having two passages, a leaf spring and a spring/passage member integrator. The chest piece has a cavity covered by a diaphragm or diaphragm assembly. Each passage is connected at one end to the cavity in the chest piece and at its other end to an earpiece. The passages embodied in the passage member are parallel for part of its length and diverge so that the member is Y-shaped in plan view. The divergent portions are appropriately shaped and positioned by arms of the spring.

The passage member is made of highly pliable, moderately elastic elastomeric material. Alternately, the passage member incorporates features which attenuate extraneous sounds entering the passage.

The invention is described in more detail below with reference to the attached drawings.

### BRIEF DESCRIPTION OF THE DRAWINGS

FIG. 1 is a general view of one embodiment of the subject stethoscope.

FIG. 2 is an exploded view of the subject stethoscope.

FIG. 3 is a section taken at 3-3 in FIG. 1.

FIG. 4 is a section taken at 4-4 in FIG. 1.

FIG. 5 is an enlarged view of parts 18 and 19 shown in FIGS. 1 and 2.

FIG. 6 illustrates a passage member to earpiece adaptor.

FIG. 7 is a sectional view illustrating details of the end of the passage member which attaches to the chest piece.

### DETAILED DESCRIPTION OF THE INVENTION

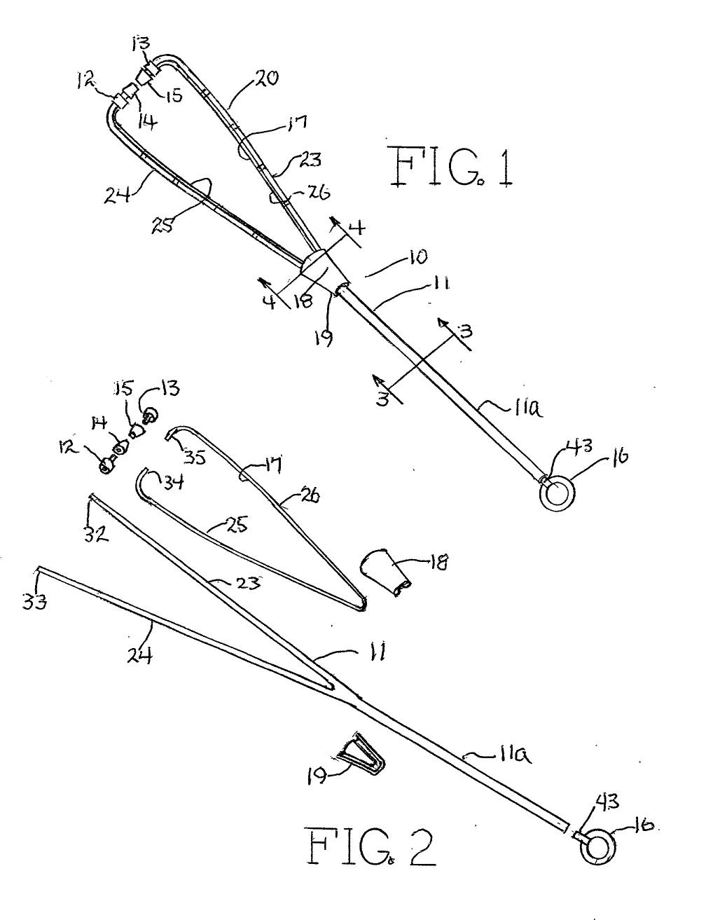

The subject invention is a dual passage, binaural stethoscope. FIG. 1 is a general view of one embodiment of the subject stethoscope 10. It comprises passage member 11, earpiece adaptors 12 and 13, earpieces 14 and 15, chest piece 16, spring 17 and integrating parts 18 and 19. These parts physically interconnect the spring and passage member. The spring and passage member may be further integrated by adhesive and/or mechanical connection using clips, clip 20 being typical. Parts 18 and 19 may be one part, molded in place. If not, they are held in place by adhesive. The passage member is bifurcated and has a non-bifurcated portion 11a and bifurcated portions 23 and 24 Spring 17 has two arms, 25 and 26.

FIG. 2 is an exploded view of the stethoscope, with parts numbered as in FIG. 1.

FIG. 3 is a section taken at 3-3 in FIG. 1, showing passages 21 and 22 molded into the passage member. In an alternate embodiment there are two passage members which are separate tubes. In another embodiment the tubes may be held side-by-side for a portion of their lengths by a covering providing a bifurcated passage member having a non-bifurcated portion and two bifurcated portions.

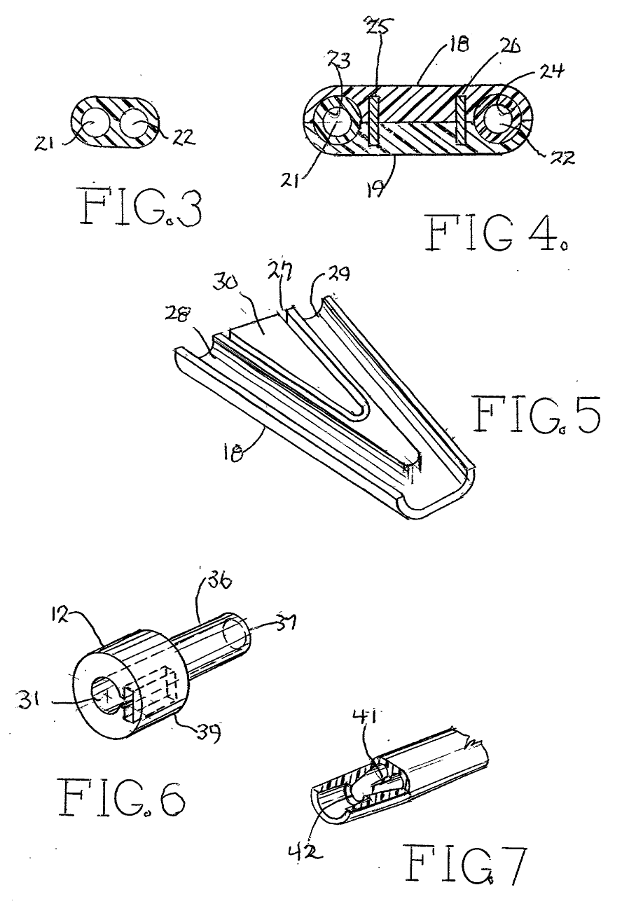

FIG. 4 is a section taken at 4-4 in FIG. 1, showing integrator parts 18 and 19 adhesively assembled and holding portions 23 and 24 of the passage member containing passages 21 and 22 and arms 25 and 26 of the spring.

FIG. 5 is an enlarged view of integrator part 18, part 19 being identical. Groove 27 accommodates the spring. Grooves 28 and 29 accommodate portions 23 and 24 of the passage member and contain passages 21 and 22. Parts 18 and 19 (or a single part that combines them) are made of stiff, hard plastic or are made of metal. Alternately parts 18 and 19 can be assembled using a screw fastener (not shown) installed in area(s) 30 of the parts.

FIG. 6 illustrates in more detail passage member to earpiece adaptors 12 and 13, 12 being shown. Cavity 31 is shaped to accommodate the ends 32 and 33 of portions 23 and 24 which are circular in cross section and ends 34 and 35 of the spring. Portion 36 of the adaptor is configured to accept earpieces of choice. The ends of portions 23 and 24 extend through the adaptor to end 37. The spring ends extend into portion 39 of the adaptor.

FIG. 7 is a sectional view illustrating details of the end 40 of the passage member to which the chest piece is attached. Portion 41 which separates passages 21 and 22 gradually diminishes so that the passages are blended into a circular cross section hole 42 which has a cross section area equal to or greater than the combined cross section areas of the passages. The stem 43 of the chest piece fits into the hole to connect the passage member to the chest piece.

It is considered to be understandable from this description that the subject invention meets its objectives. It provides a stethoscope having dual passage, binaural, pneumatic sound transmission. It is also competitive in terms of performance, weight, durability and professional quality and appearance.

It is also considered to be understood that while certain embodiments of the invention are disclosed herein, other embodiments and modifications of those disclosed are possible within the scope of the invention which is limited only by the attached claims.

I claim:

1. A dual passage, binaural stethoscope, said stethoscope comprising:
a chest piece,
at least one passage member having first and second ends and first and second passages, each of which extends from said first end to said second end, thus having first and second passage ends,
a leaf spring having first and second spring ends and
first and second earpieces,
said first earpiece being connected to said first end of said first passage,
said second earpiece being connected to said first end of said second passage,
said first earpiece and said first end of said first passage being attached to said first spring end and said second earpiece and said first end of said second passage being connected to said second spring end,
said second ends of said first and second passages being connected to said chest piece.

2. The stethoscope of claim 1 having first and second passage members, each of said passage members being a flexible tube, said first passage being in said first passage member and said second passage being in said second passage member.

3. The stethoscope of claim 2 in which each of said passage members has lengthwise first and second portions and said spring has first and second arms, said passage members being joined side-by-side over said second portions and forming a juncture between said first and second portions, said first portion of said first passage member being connected to said first arm and said first portion of said second passage member being connected to said second arm.

4. The stethoscope of claim 3 further comprising at least one integrator part, installed at said juncture and integrating said first and second portions of said passage members and said spring.

5. The stethoscope of claim 1 having one passage member, said member being bifurcated and having a non-bifurcated portion and first and second bifurcated portions and a juncture between said bifurcated and non-bifurcated portions, said first passage extending through said non-bifurcated portion and said first bifurcated portion, said second passage extending through said non-bifurcated portion and said second bifurcated portion, said stethoscope further comprising at least one integrator part installed at said juncture and integrating said spring, said non-bifurcated portion and said bifurcated portions.
